(19) United States
(12) Patent Application Publication
(10) Pub. No.: US 2025/0177439 A1
(43) Pub. Date: Jun. 5, 2025

(54) LOW DOSE PRE-MIX FEED FOR TRACE MINERAL SUPPLEMENTATION

(71) Applicant: Chemlock Operating LLC d/b/a Chemlock Nutrition, West Chester Township, OH (US)

(72) Inventors: Joshua Ryan Steed, Winder, GA (US); Arun Mehan, Mason, OH (US); Grant Thomas Shouldice, Mason, OH (US)

(73) Assignee: Chemlock Operating LLC d/b/a Chemlock Nutrition, West Chester Township, OH (US)

(21) Appl. No.: 18/964,087

(22) Filed: Nov. 29, 2024

Related U.S. Application Data
(60) Provisional application No. 63/605,298, filed on Dec. 1, 2023.

(51) Int. Cl. A61K 33/30; A61K 33/32; A61P 3/02

(52) U.S. Cl. CPC A61K 33/30; A61K 33/32; A61P 3/02

(57) ABSTRACT

The present disclosure relates to compositions and methods for maintaining or improving the health of an animal. The invention provides for animal or livestock feeds and supplements and particularly to animal feeds and supplements comprising a low dose formulation of zinc and manganese from a covalently bonded source heretofore designated as a hydroxy chloride trace mineral source (HTM), which leads to improved health in animals. The invention also provides for animal feeds and supplements and particularly to animal feeds and supplements containing a low dose formulation of zinc and manganese from a covalently bonded source heretofore designated as a hydroxy chloride trace mineral source (HTM), while maintaining a controlled level of copper and/or phytase in the common basal feed mix.

### CROSS-REFERENCE TO RELATED APPLICATIONS

This application claims the benefit of priority under 35 U.S.C. § 119(e) to U.S. Patent Application Provisional Application Ser. No. 63/605,298, entitled “LOW DOSE PRE-MIX FEED FOR TRACE MINERAL SUPPLEMENTATION,” filed Dec. 1, 2023, the contents of which are hereby incorporated by reference in their entirety for any purpose.

### BACKGROUND

### 1. Technical Field

The present invention relates to animal and bird nutritional supplementation, in particular poultry, of trace minerals in a highly bioavailable, absorbable, format with a novel low dose methodology and premix feed composition.

### 2. Description of the Related Art

The present invention is broadly concerned with a method of supplementation, including but not limited to the physiochemical state and dose level of essential metal elements in novel quantities, and in an enhanced biologically available form in the diet, for increasing the health and well-being of domestic animals and birds and in particular domestic poultry. The history of the amazing discoveries of the roles that trace minerals play in nutrition in all species and especially in livestock is as extensive as it is fascinating. (Suttle, 2010) Over the last three to four centuries it has become widely known that all animal and plant tissues contain varying amounts of mineral elements and tend to exist in the oxide, phosphate, carbonate and sulfate forms. (Suttle, 2010) By the early 1980s, it was shown that twenty-two minerals were essential for animal life. Seven minerals including calcium, phosphorus, potassium, sodium, chlorine, magnesium, and sulfur were designated as major or macronutrients. Fifteen mineral elements including iron, iodine, zinc, copper, manganese, cobalt, molybdenum, selenium, chromium, tin, vanadium, fluorine, silicon, nickel and arsenic were designated as trace or micronutrients. (Underwood, 1977) In fact, it was shown that several proteins require a metal as a cofactor thereby making metal ions essential for most forms of life. (Suttle, 2010) Furthermore, elements including cobalt, copper, iron, iodine, manganese, molybdenum, selenium and zinc among others are essential for the normal functioning of a body's metabolic functions. (Underwood, 1977) (López-Alonso, 2012) Moreover, optimized, and adequate trace minerals facilitate some of the most important structural, physiological, catalytic, and regulatory processes in the body. This is due, in large part, to this group of transition metal's ability to exist in an ionic state or covalently bound to certain atoms thereby spawning the source (i.e. the physiochemical state as it is consumed) of the trace minerals as one of the richest areas of academic and commercial research. (Suttle, 2010) (Weller, Overton, Rourke, & Armstrong, 2014) (Brugger & Windisch, 2015) Prior art with respect to the source, of the trace mineral focused on the physiochemical state, the full diet and supplementation. Once ingested by an organism in whatever physiochemical form, the homeostasis mechanism, including absorption by the body, agonistic and antagonistic effects as well as other chemical interactions and their effect on bioavailability and excretion, was and continues to be, another broad area of study. (Suttle, 2010) (Underwood, 1977) The corollary of the knowledge generated in the areas of a) trace minerals source and b) the homeostasis mechanistic research, is a good understanding and quantification of the levels and rates of ingestion including the effects of deficiency and surplus of trace minerals. Source, homeostasis, and optimal level studies exist in the public domain for all trace elements and for all species in what is an enormous body of nutritional research. The scope of the art taught in this patent necessitates a review of a much narrower list of trace mineral elements in particular copper, zinc, and manganese. Furthermore, with respect to the organism of interest, the background review scope narrows to domestic poultry nutrition.

The trace mineral elements of copper, zinc and manganese were shown to be extremely important to the health and well-being of all animals including domestic poultry which comprises chickens, both broilers and layers, and turkeys. (Suttle, 2010) (Underwood, 1977) (Prasad, 1967) (Olukosi, van Kuijk, & Han, 2018) The activity of numerous enzymes, cofactors and reactive proteins depends on the presence of copper making it essential. (Suttle, 2010) Hemoglobin formation depends on essential copper and it also plays a role in for example cytochrome oxidase, which is important in oxidative phosphorylation. (Leeson, 2009) Copper also has a role in the crosslinking of collagen and elastin which gives bone its tensile strength and elasticity. (Dibner, Richards, Kitchell, & Quiroz, 2007)

The essentiality of copper for processes such as reproduction and bone development depend on its participation in such functions. Zinc, on the other hand, is a required constituent of several hundred metalloenzymes and therefore impacts a wide array of basic cellular functions. (Suttle, 2010) However, a list of zinc metalloenzymes would only partially cover zinc's importance given the sheer number zinc functions. Zinc is required for the structural and functional integrity of over two thousand transcription factors and almost every signaling and metabolic pathway is dependent on one or more zinc-requiring proteins. (Beattie & Kwun, 2004) (Cousins, Liuzzi, & Lichten, 2006) Tetrahedral coordination of zinc to cysteine and histidine residues creates ‘zinc-finger’domains in DNA-binding proteins. (Berg, 1990) The most important functions in livestock are those that become limiting to health and production when they are deprived of zinc. There are four outstanding, interconnected candidates. The element participates in regulation of hydroxyapatite crystallization, collagen synthesis, and the cellular invasion of the cartilage matrix by the osteoblasts. (Dibner, Richards, Kitchell, & Quiroz, 2007) Manganese also plays both structural and catalytic roles in metalloproteins (Scrutton, Utter, & Mildvan, 1966) It is necessary for the normal development of bone. The ground substance of tissue, particularly the proteoglycan matrix in which collagen and elastin are embedded, requires manganese for glycosylation of its protein core molecule. (Dibner & Richards, 2005) A second function was identified when manganese superoxide dismutase was isolated from chicken liver mitochondria and subsequently found to contain 2 mg Mn/mol. (Gregory & Fridovich, 1974) Manganese superoxide dismutase complements the copper version in protecting cells from damage by reactive oxygen species, notably the superoxide radical O₂·⁻. It has been suggested that manganese superoxide dismutase is stress-responsive and needed for added protection against oxidative stress associated with inflammatory responses to some infections. (St Clair, 2004) Interestingly enough, compensatory increases in the copper super oxide dismutase suggested overlapping roles and possible interactions between dietary copper and manganese. The essentiality of copper, zinc and manganese is well established above however, full treatment can be found in the references herein. Prior art that discloses source of the element and the levels required for proper nutrition are discussed next.

There are various sources of the essential elements copper, zinc, and manganese. From a broad perspective, the sources can be categorized as ionic, organic, and covalent. As mentioned above, ionic sources include sulfates, carbonates, phosphates and oxides and are referred to in the collective as ionic trace minerals (ITM). Organic sources are typically metal amino acids or chelates of amino acids (e.g. methionine chelates) and are referred to in the collective as organic trace minerals (OTM). Finally, the covalent bonded metals in the form of the alkaline base are referred to in the collective as hydroxy trace minerals (HTM) (e.g. basic copper chloride).

Studies in the 1990s revealed that a trace mineral source (Cu or Zn) linked to an alkaline base form a hydroxychloride. The hydroxychloride is formed by covalent bonds that are resistant to neutral pH conditions, and the release of trace minerals occurs under acid pH conditions. (Cohen, 2014) According to Pang and Applegate, hydroxychloride shows low solubility under basic pH conditions in vitro when compared to sulphate. (Pang & Applegate, 2007) Their result suggested that hydroxychlorides could resist passage through the first organs of the gastrointestinal tract, thus avoiding negative interactions with other nutrients and facilitating absorption in the small intestine.

Recently, Dos Santos et al. disclosed the effects of dietary copper and zinc hydroxychloride supplementation on bone development, skin quality and hematological parameters of broilers chickens. (dos Santos, et al., 2023) They found that the hematological parameters were not influenced by mineral supplementation in Cobb-500 Broilers. However, the inclusion of 105-140 ppm zinc form zinc hydroxy chloride enhanced the skin strength compared to 132-177 pmm of zinc from zinc hydroxy chloride. The study also claimed that bone mineral density of the tibia proximal epiphysis, tibia ash and tibia mineral content were positively improved with supplementation of 15-29 ppm of Cu for basic copper chloride and 109-139 ppm of zinc from zinc hydroxy chloride compared to 100-177 ppm of copper from basic copper chloride. This study demonstrated that hydroxy compounds are potential alternatives for replacing sulphate supplements in broiler diets. Moreover, among the Cu and Zn levels, 15 ppm of copper from basic copper chloride and 100 pm of zinc from zinc hydroxy chloride improved bone development and skin integrity, suggesting that the combination of Cu and Zn can be a nutritional strategy to prevent the incidence of leg disorders in broilers.

In another study, the effect of feeding broilers with inorganic, organic and coated trace minerals on the performance, economics and retention of copper and zinc was articulated by Lu et al. (Lu, Kuang, Ma, & Liu, 2020). The authors disclosed, using a classic feeding methodology, that there were no improvements with organic metal methionine vs ITM supplementation. However, statistically significant improvements were determined in the overall performance of the broilers when fed six trace minerals including copper, zinc and manganese coated using a proprietary carbohydrate mixture. In this study copper was fed at 4-5 ppm and zinc and manganese were fed at 36-48 ppm. They found that the organic forms and the coated ITM performed better than ITM alone with respect to weight gain, feed intake and metal retention.

In 2021, the production method of the poultry premix coated with the slow-release trace elements was taught by CN CN113598281A. (Inventors [symbol] 2021) The trace elements were covered by a coating comprising the following steps: an outer gastric coating layer and an inner pellet core; the gastric coating layer (coating material) of the outer layer comprises dextrin, xanthan gum, fossil powder, carboxymethyl cellulose, arabic gum, sodium alginate, polylactic acid and polyacrylic acid. The core of the inner layer comprised trace elements, essential oil for preventing gastroenteritis, and essential oil for increasing feed intake; the trace elements include zinc sulfate, glycine iron, copper sulfate, manganese sulfate, sodium selenite and calcium lactate. It is not clear from the invention what benefit to the animal or producer of the animal achieved or what levels of the trace mineral were in the feed composition.

In another invention, a feed additive for an animal, including base of trace elements from 7.4%-86.7% and an acceptable filler between 13.3% to 92.6 where the base of trace elements included: iron, manganese, zinc, copper, cobalt in the form of complex compounds with L-aspartic acid in a molar ratio of amino acid-metal-2:1; among other nutritional components. (Inventors: [symbol] , 2020). It is not clear from the invention what benefit to the animal or producer of the animal achieved or what levels of the trace mineral were in the feed composition.

In another study, the effect of supplemental Mn, Zn, Fe and Cu and their interactions on the performance of broiler chickens was disclosed. (Holubiev, Holubieva, & Sychov, 2020) They found that the use of mixed feeds in feeding broilers chicken which contained the glycinate of manganese (75-100 ppm), zinc (75-100 ppm), iron and copper (11-15 ppm) at 75% of the needs, contributed to increasing their body weight by 2.5% and increasing the growth rate by 2.4%.

In another study, Olukosi et al. disclosed the effect of supplementation of sulfate or hydroxychloride forms of Zn and Cu (15 ppm) at two supplemental Zn levels (20 ppm vs 80 ppm) on growth performance, meat yield, and tissue levels of Zn. (Olukosi, van Kuijk, & Han, 2018) They found that broiler chickens receiving hydroxychloride Zn and Cu had greater (P<0.05) gains. Broiler chickens receiving lower Zn level (20 ppm) had greater (P<0.01) weight gain as well. Moreover, broiler chickens receiving hydroxychloride Zn and Cu had greater (P<0.05) % breast yield than those receiving sulfate Zn and Cu. Higher level of Zn (80 ppm), irrespective of source, produced greater (P<0.01) tibia and plasma Zn levels, whereas liver Cu was greater (P<0.05) in broiler chickens receiving hydroxy chloride Zn and Cu. It was concluded that hydroxy chloride Zn and Cu were more efficacious than sulfate Zn and Cu in promoting growth performance and enhancing meat yield. Manganese was not considered in this particular study.

In another study published in 2015, the effects of dietary supplementation of organic and inorganic Mn, Zn, Cu, and Cr mixtures using two different levels (80, 60, 5, and 0.15 mg/kg and 40, 30, 2.5, and 0.07 mg/kg, respectively) on the bioavailability of these trace minerals and Ca in late-phase laying hens were evaluated. (Yenice, Mizrak2, Gülteki, & Tunca, 2015) They found that the organic form significantly increased the concentrations of serum Mn, Zn, Cu, and Ca as well as egg Mn, Zn, Cu, and Cr and eggshell Zn and Cr compared with the inorganic form. However, the form of trace minerals did not affect the concentrations of serum Cr and eggshell Mn, Cu, and Ca. High-level addition of trace minerals significantly increased serum Mn and Zn; egg Mn, Zn, Cu, and Cr; and eggshell Mn, Zn, and Cu concentrations compared with low level addition but did not affect serum Cu, Cr, and Ca or eggshell Cr and Ca concentrations. While the organic form reduced the excretion of Mn, Zn, Cu, Cr, and Ca, the high level supplement increased Mn, Zn, and Cu excretion. The addition level did not affect Cr and Ca excretion. These results demonstrated that dietary supplementation of an organic Mn, Zn, Cu, and Cr mixture increases the bioavailability of Mn, Zn, Cu, Cr, and Ca compared with inorganic sources and that a lower level of trace mineral supplementation results in lower mineral excretion, particularly in an organic form.

It is readily apparent from vast body of prior art above and references therein that hydroxy trace minerals are a superior supplementation form. However, to date, a feed method that considers low dose supplementation using hydroxy trace minerals for copper, zinc, manganese, trace minerals from plant based standard ration of silage, the amount to phytate as well as the amount of phytase in the feed has not been disclosed or taught in the prior art.

### BRIEF SUMMARY OF THE INVENTION

In one aspect, the present disclosure provides compositions and methods for trace mineral supplementation in livestock animals.

The present invention relates to a feed method and feed mixture for poultry which utilizes a heretofore unknown and novel low dose supplementation of zinc and manganese from a covalently bonded source heretofore designated as a hydroxy chloride trace mineral source (HTM) while maintaining a controlled level of copper and phytase in the common basal feed mix.

Zinc chloride hydroxide monohydrate is a zinc hydroxy compound with chemical formula Zn₅(OH)₈Cl₂·H₂O. It is often referred to as tetrabasic zinc chloride (TBZC), basic zinc chloride, zinc hydroxychloride, or zinc oxychloride.

Manganese hydroxychloride is identified by the Chemical Abstracts Service (CAS) No. 39438-40-9. Its molecular formula is Mn₂(OH)₃Cl and it has a molecular weight of 196.35 Da. Other names are tribasic manganese chloride (TBMC) and basic manganese chloride.

In one embodiment, the present invention provides for methods of supplementing feed with the use of a zinc supplementation in the form of tetrabasic zinc hydroxy chloride (TBZC) and a manganese supplementation in the form of tribasic manganese hydroxy chloride (TBMC) to a livestock diet.

In one embodiment, the present invention provides for methods of supplementing feed with the use of a 1-40 ppm zinc supplementation in the form of tetrabasic zinc hydroxy chloride and a 1-50 ppm manganese supplementation in the form of tribasic manganese hydroxy chloride to a poultry diet.

In one embodiment, the present invention provides for methods of supplementing feed with the use of a 5-30 ppm zinc supplementation in the form of tetrabasic zinc hydroxy chloride and a 5-40 ppm manganese supplementation in the form of tribasic manganese hydroxy chloride to a poultry diet.

In another embodiment, the present invention provides for methods of supplementing feed with the use of a 10-30 ppm zinc supplementation in the form of tetrabasic zinc hydroxy chloride and a 20-40 ppm manganese supplementation in the form of tribasic manganese hydroxy chloride to a poultry diet.

In another embodiment, the present invention provides for methods of supplementing feed with the use of a 20-30 ppm zinc supplementation in the form of tetrabasic zinc hydroxy chloride and a 30-40 ppm manganese supplementation in the form of tribasic manganese hydroxy chloride to a poultry diet.

In another embodiment, the feed comprises at least 1, 2, 3, 4, 5, 6, 7, 8, 9, 10, 11, 12, 13, 14, 15, 16, 17, 18, 19, 20 or more ppm zinc supplementation in the final feed. In another embodiment, the feed comprises at least 1, 2, 3, 4, 5, 6, 7, 8, 9, 10, 11, 12, 13, 14, 15, 16, 17, 18, 19, 20 or more ppm manganese supplementation in the total weight of feed. In another embodiment, the feed comprises at most 40, 35, 30, 25, or less ppm zinc supplementation in the final feed. In another embodiment, the feed comprises at most 50, 45, 40 or less ppm manganese supplementation in the total weight of feed.

In one embodiment, the present invention provides for methods of supplementing feed with the use of a 25 ppm zinc supplementation in the form of tetrabasic zinc hydroxy chloride and a 37.5 ppm manganese supplementation in the form of tribasic manganese hydroxy chloride to a poultry diet comprised of a common basal whose major bulk ingredients used are corn, soybean meal with a 3% fixed inclusion in all phases of meat and bone meal.

In another preferred embodiment, the supplementation and diet are fixed for all four growth phases, which are known to those well versed in the art of poultry husbandry.

Poultry feed in the present invention may also include additional active ingredients such as antioxidants, anti-inflammatories, anti-hyperlipidemics, probiotics, prebiotics, vitamins, minerals, amino acids, vasodilators, phospholipase A2 inhibitors (PLA2), and phytases.

In another preferred embodiment, the poultry diet includes a coccidiostat (Elanco, Coban 90), 500 FTU/kg of Phytase (AB Vista, Quantum Blue 5G), an NSPase (AB Vista, Econase XTP) and 125 ppm of copper in the form of tribasic copper chloride (Chemlock Nutrition, Nutrilock® 58% TBCC). Tribasic copper chloride (TBCC), refers to the compound with chemical formula Cu₂(OH)₃Cl. It is often referred to as dicopper chloride trihydroxide, copper trihydroxyl chloride or copper hydroxychloride. Compared to copper sulfate, the alpha crystal form of basic copper chloride provides many benefits including improved feed stability, less oxidative destruction of vitamins and other essential feed ingredients; superior blending in feed mixtures, and reduced handing costs. It has been widely used in feed formulations for most species, including chickens, turkeys, pigs, beef and dairy cattle, horses, pets, aquaculture and exotic zoo animals.

In another preferred embodiment, amino acid ratios were controlled and followed the genetic and USA industry standards.

In another preferred embodiment, trace mineral premixes were included at 0.100% of the complete feed and mixed with a common basal in a standing Hobart mixer for 15 minutes for incorporation prior to large scale mixing and pelleting of each treatment.

The additive in the invention may assume any physical form that is appropriate to be mixed into poultry feed, such as powder, granules, bran, pellets, etc.

Another aspect of the invention refers to materials and methods that provide for animal or livestock feeds and supplements and particularly to animal feeds and supplements comprising a low dose formulation of zinc and manganese from a covalently bonded source heretofore designated as a hydroxy chloride trace mineral source (HTM), which leads to improved health in animals.

The above summary contains simplifications, generalizations and omissions of detail and is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed written description.

### BRIEF DESCRIPTION OF THE DRAWINGS

The description of the illustrative embodiments can be read in conjunction with the accompanying figures. It will be appreciated that for simplicity and clarity of illustration, elements illustrated in the figures have not necessarily been drawn to scale. For example, the dimensions of some of the elements are exaggerated relative to other elements. Embodiments incorporating teachings of the present disclosure are shown and described with respect to the figures presented herein, in which:

FIG. 1 illustrates Table 1 showing Zero to 14 day growth stage data: Body weight, body weight gain, feed to gain, and feed intake in broilers fed different trace mineral premixes from 0 to 14 days of age.

FIG. 2 illustrates Table 2 showing processing yields from 50 day broilers fed different trace mineral premixes from 0-49 days of age.

### DETAILED DESCRIPTION

### Definitions

Embodiments described below in context of the user input devices are analogously valid for the respective methods, and vice versa. Furthermore, it will be understood that the embodiments described below may be combined, for example, a part of one embodiment may be combined with a part of another embodiment.

It should be understood that the singular terms “a”, “an”, and “the” include plural references unless context clearly indicates otherwise. Similarly, the word “or” is intended to include “and” unless the context clearly indicates otherwise.

The terms “metal” and “mineral” may be used interchangeably. Each refers particularly to any divalent or trivalent metal that, when in ionic form, can form one or more coordinate bonds with a ligand, and is substantially non-toxic when administered in traditional amounts as known in the art. The metal is preferably a metal selected from the group consisting of Cu, Mn, Mg, Fe, Zn, Cr, and Ca. In one embodiment, the metal is selected from the group consisting of Cu, Mn, and Zn.

The term “amino acids” or “naturally occurring amino acids” shall mean alpha-amino acids that are known to be used for forming the basic constituents of proteins, including alanine, arginine, asparagine, aspartic acid, cysteine, cystine, glutamine, glutamic acid, glycine, histidine, hydroxyproline, isoleucine, leucine, lysine, methionine, ornithine, phenylalanine, proline, serine, threonine, tryptophan, tyrosine, valine, and combinations thereof.

When referring to “metal salts” or “salts,” it is recognized that oxides and hydroxides are not technically salts in the classic sense. However, in accordance with embodiments of the present invention, metal oxides and hydroxides are considered to be salts along with any other metal salts as more typically defined.

As used herein, the term “livestock” includes warm-blooded animals kept or raised for use or pleasure. Typically, “livestock” refers to animals that are commonly kept or raised for some commercial use or purpose. These may be animals that are kept in confinement within a building or shelter, or within some partially or fully enclosed area of land. Alternatively, livestock may be allowed to roam freely over an open area of land. In one specific embodiment, “livestock” refers to animals selected from the group consisting of swine, ruminants, poultry, equines, and any combination thereof. In one or more embodiments, the livestock is poultry. In one or more embodiments, the poultry is a chicken, turkey, guinea fowl, duck, goose, pigeon or quail in all growth stages.

The term “supplement” as used herein shall mean any foodstuff, composition, or compound that contains a substance intended to benefit an animal, and is provided to the animal in order to increase the amount of that substance ingested by the animal above the amount it receives by its normal dietary behavior.

As used herein, the term feed material refers to the basic feed material to be consumed by an animal. It will be further understood that this may comprise, for example, at least one or more unprocessed grains, and/or processed plant and/or animal material such as corn, soybean meal or bone meal. In some embodiments, the feed material will comprise one or more of the following components: a) cereals, such as small grains (e.g., wheat, barley, rye, oats and combinations thereof) and/or large grains such as maize or sorghum; b) by products from cereals, such as corn gluten meal, Distillers Dried Grain Solubles (DDGS), wheat bran, wheat middlings, wheat shorts, rice bran, rice hulls, oat hulls, palm kernel, and citrus pulp; c) protein obtained from sources such as soya, sunflower, peanut, lupin, peas, fava beans, cotton, canola, fish meal, dried plasma protein, meat and bone meal, potato protein, whey, copra, sesame; d) oils and fats obtained from vegetable and animal sources; e) minerals and vitamins.

As used herein, the term feedstuff refers to a feed material to which one or more feed supplements have been added. It will be understood by the skilled person that different animals require different feedstuffs, and even the same animal may require different feedstuffs, depending upon the purpose for which the animal is reared. It will be further understood that depending on the starting feed material, the feedstuff may be a high fiber feedstuff or a low fiber feedstuff.

The feedstuff may be a feedstuff for a monogastric animal, such as poultry (for example, broiler, layer, broiler breeders, turkey, duck, geese, waterfowl), swine (all age categories), a pet (for example dogs, cats) or fish.

As used herein, “monogastric” is intended to encompass any animal having one stomach. Examples of monogastric animals include, but are not limited to, fish, horses, emu, ostrich, dog, cat, swine, bear, turkey, chickens, ducks, geese, quail, pheasants, reptiles, waterfowl, and humans. Pre-ruminant animals such as young cattle, buffalo, bison, and elk are also encompassed by the term monogastric as these animals are born monogastric and then develop into true ruminants as adults.

In one embodiment, the feedstuff may comprise feed materials comprising maize or corn, wheat, barley, triticale, rye, rice, tapioca, sorghum, and/or any of the by-products, as well as protein rich components like soybean mean, rape seed meal, canola meal, cotton seed meal, sunflower seed mean, animal-by-product meals and mixtures thereof. More preferably, the feedstuff may comprise animal fats and/or vegetable oils.

Optionally, the feedstuff may also contain additional minerals such as, for example, calcium and/or additional vitamins. In one embodiment, calcium may be added to the feedstuff in any suitable amount to supplement the diet of the animal and/or as a bulking agent. The calcium may be in the form of organic (e.g., leafy green vegetables) or inorganic (e.g., limestone/calcium carbonate) calcium. In one embodiment, after addition of the calcium supplement, the feedstuff will comprise at least about 0.5% calcium. In another embodiment, at least about 0.8%, at least about 1.0%, at least about 2.0%, at least about 3% calcium.

The feedstuff may comprise at least 0.0005% by weight of the feed supplement. In one embodiment, the feedstuff may comprise at least 0.0005%; at least 0.0010%; at least 0.0020%; at least 0.0025%; at least 0.0050%; at least 0.0100%; at least 0.020%; at least 0.100% at least 0.200%; at least 0.250%; at least 0.500% by weight of the feed supplement. In one embodiment, the feedstuff may comprise from about 0.001% to about 0.5% by weight of the feed supplement. In another embodiment, the feedstuff may comprise from about 0.02% to about 0.5% by weight of the feed supplement. In another embodiment, the feedstuff may comprise from about 0.05% to about 0.25% by weight of the feed supplement. In another embodiment, the feedstuff may comprise from about 0.05% to about 0.2% by weight of the feed supplement.

It will be apparent to the skilled person that the feed supplement of the present invention will be particularly beneficial when used in a feedstuff comprising high calcium. It will be apparent to a skilled person that the level of calcium which represents a high calcium diet may vary depending on the type of animal and even the use for which the animal is reared. For example, for laying hens, a diet with greater than about 3% would be high Ca diet. For broilers and all turkeys, a diet with >1% Ca would be high Ca diet. For the purposes of the present invention, a high calcium diet is considered to be a diet comprising at least 1-2% calcium.

Phytate (or phytic acid also known as inositol hexaphosphate, or IP6) is a compound found in plant-based foods that stores phosphorus and minerals. It's the salt form of phytic acid, which is the primary way plants store phosphorus. Phytate is present in many foods, including grains such as whole wheat, oats, and rice.

The majority of phosphorus (P) in cereal grains is bound in the form of phytate, which has poor biovailability to monogastrics. Therefore, microbial phytases are widely used in poultry feeds as a means of improving dietary P availability and reducing P excretion in manure. Phytase is an enzyme that releases phosphorus from phytate, a storage form of phosphorus in plant-based feedstuffs. Phytase can improve phosphorus digestibility and reduce the antinutritional effects of phytate.

The term “phytase” as used herein means any type of phosphatase enzyme that catalyzes the hydrolysis of phytic acid (myo-inositol hexakisphosphate)—an indigestible, organic form of phosphorus that is found in many plant tissues, especially in grains and oil seeds—and releases a usable form of inorganic phosphorus.

Phytase FTU/kg is a measurement of phytase activity in a feed, where FTU stands for phytase units:
FTU=The amount of enzyme that releases 1 kmol of inorganic phosphate from phytate per minute
FTU/kg=A measurement of phytase activity in a feed

The optimal dose of phytase for phosphorus liberation can vary depending on the price of inorganic phosphorus and other factors. For example, the optimum dose of phytase for poultry is about 1000 FTU/kg, but could range from 500-1500 FTU/kg depending on price conditions.

The term “NSPase” as used herein means any type of non-starch polysaccharide (NSP)-degrading enzymes, which are added to diets of food producing animals to increase the energy obtained from diets with high fiber content. NSP-degrading enzymes include enzymes such as arabinanase, cellulases, glucanases, xylanases, and pectinases. NSPase enzymes are generally multi-enzyme based on the non-starch polysaccharide composition, content, and the digestibility and absorption of feed nutrients. It primarily contains xylanase, beta-glucanase, cellulanase and mananase, etc. NSPase enzymes can break down the cell walls, reduce the viscosity of digesta, and then enhance the utilization of energy, protein, amino acids and mineral element in the feedstuff. Non-starch polysaccharide (NSP) degrading enzymes (NSPases), such as xylanase or xylanase-based enzymatic complexes, are of special interest to optimize animal production, as their use will bring an economic advantage via increased zootechnical performance or via lower feed costs (due to the ability of the enzymes to improve the metabolizable energy content of the feed).

The term “coccidiostats” as used herein means antiprotozoal agents that act on coccidia parasites by inhibiting reproduction and retarding the development of the parasite in a host cell. They are most commonly used in poultry populations by addition in the feed at the authorized levels and observing the prescribed hygiene requirements. The disease can also occur in other food-producing animals including pigs, calves, and lambs.

It will be readily apparent to the skilled person that in order for the feed supplement of the present invention to provide the claimed advantages, the formulation of zinc and manganese must be present in the feed material or feedstuff. It will also be readily apparent that this formulation of zinc and manganese may be present naturally as a constituent of the feed material, or may be added as an additional supplement at a desired level.

In another aspect there is provided a method for producing a feedstuff. Feedstuff is typically produced in feed mills in which raw materials are first ground to a suitable particle size and then mixed with appropriate additives. The feedstuff may then be produced as a mash or pellets; the later typically involves a method by which the temperature is raised to a target level and then the feed is passed through a die to produce pellets of a particular size. The pellets are allowed to cool. Subsequently liquid additives such as fat and enzyme may be added. Production of feedstuff may also involve an additional step that includes extrusion or expansion prior to pelleting, particular by suitable techniques that may include at least the use of steam.

### Detailed Description of Exemplary Embodiments

The present disclosure relates to compositions and methods for maintaining or improving the health of an animal.

In one or more embodiments, the present invention provides for animal or livestock feeds and supplements and particularly to animal feeds and supplements containing a low dose formulation of zinc and manganese from a covalently bonded source heretofore designated as a hydroxy chloride trace mineral source (HTM), which leads to improved health in animals.

In one or more embodiments, the present invention provides for animal feeds and supplements and particularly to animal feeds and supplements containing a low dose formulation of zinc and manganese from a covalently bonded source heretofore designated as a hydroxy chloride trace mineral source (HTM), while maintaining a controlled level of copper and/or phytase in the common basal feed mix.

In one embodiment, the present invention provides for animal or livestock feeds and supplements comprising a final concentration of 1-30 ppm zinc supplementation in the form of tetrabasic zinc hydroxy chloride and a 1-40 ppm manganese supplementation in the form of tribasic manganese hydroxy chloride to a poultry diet comprised of a common basal. In one embodiment, the common basal comprises major bulk ingredients used are corn and/or soybean meal.

In one embodiment, the present invention provides for methods of supplementing feed with the use of a 1-30 ppm zinc supplementation in the form of tetrabasic zinc hydroxy chloride and a 1-40 ppm manganese supplementation in the form of tribasic manganese hydroxy chloride to a poultry diet. In another embodiment, the present invention provides for methods of supplementing feed with the use of a 10-30 ppm zinc supplementation in the form of tetrabasic zinc hydroxy chloride and a 20-40 ppm manganese supplementation in the form of tribasic manganese hydroxy chloride to a poultry diet. In another embodiment, the present invention provides for methods of supplementing feed with the use of a 20-30 ppm zinc supplementation in the form of tetrabasic zinc hydroxy chloride and a 30-40 ppm manganese supplementation in the form of tribasic manganese hydroxy chloride to a poultry diet.

In one embodiment, the present invention provides for animal or livestock feeds and supplements comprising a 3% fixed inclusion in all phases of meat and bone meal.

In another preferred embodiment, the supplementation and diet are fixed for all four growth phases, which are known to those well versed in the art of poultry husbandry.

In one or more embodiments, the feed supplement comprises at least one enzyme having enzyme activities selected from the group including glucanase, xylanase, cellulase, protease, and phytase activities. In one embodiment the feed supplement comprises at least one lipolytic enzyme and/or at least one phytase and/or at least one NSPase.

In one or more embodiments, the feed supplement may comprise at least one further enzyme. In preferred embodiments, the at least one further feed enzyme is selected from the group consisting of those involved in starch metabolism, fibre degradation, lipid metabolism, proteins or enzymes involved in glycogen metabolism, acetyl esterases, aminopeptidases, amylases, arabinases, arabinofuranosidases, carboxypeptidases, catalases, cellulases, chitinases, chymosin, cutinase, deoxyribonucleases, epimerases, esterases, -galactosidases, -glucanases, glucan lysases, endo-glucanases, glucoamylases, glucose oxidases, -glucosidases, including beta glucosidase, glucuronidases, hemicellulases, hexose oxidases, hydrolases, invertases, isomerases, laccases, lyases, mannosidases, oxidases, oxidoreductases, pectate lyases, pectin acetyl esterases, pectin depolymerases, pectin methyl esterases, pectinolytic enzymes, peroxidases, phenoloxidases, polygalacturonases, proteases, rhamno-galacturonases, ribonucleases, thaumatin, transferases, transport proteins, transglutaminases, xylanases, hexose oxidase (D-hexose: O2-oxidoreductase, EC 1.1.3.5) beta-glucanase, alpha-amylase, pectinase, cellobiohydrolase, acid phosphatases and/or others or combinations thereof. These include enzymes that, for example, modulate the viscosity of the feed.

In one or more embodiments, the feed supplement comprises at least one enzyme selected from the group consisting of an amylase, an arabinoxylanase, a cellulase, a galactosidase, a hemicellulase, a lipase, a xylanase, an NSPase, a phytase, a protease, a xylanase, and combinations thereof. In another embodiment, the feed supplement comprises an NSPase, a phytase, and combinations thereof.

In one or more embodiments, the enzyme is present in the range of 10 U/kg to 10000 U/kg feed, more preferably, 100 U/kg to 7500 U/kg, and even more preferably, 200 U/kg to 5000 U/kg. In another embodiment, the enzyme is present in the range of 300 U/kg to 2500 U/kg feed. In another embodiment, the enzyme is present in the range of 500 U/kg to 1500 U/kg feed.

### Phytase

In some embodiments, the animal feeds and supplements comprising a low dose formulation of zinc and manganese from a covalently bonded source heretofore designated as a hydroxy chloride trace mineral source (HTM), also comprise a phytase.

The phytase enzyme may be derived from fungi, yeast, bacteria, protozoa, or plants, wherein the fungi or bacteria may be thermophilic. The phytase maybe an acid phytase, an alkaline phytase, a 3-phytase, or a 6-phytase. The phytase may be a wild type phytase or a modified or variant phytase comprising at least one amino acid substitution. Modified or variant phytases may have improved biochemical properties, such as improved pH activity, improved pH stability, improved thermostability, improved specific activity, improved kinetics, improved stability to proteases, and the like. The phytase may be isolated from the original organism or the phytase may be recombinantly produced (i.e., expressed in yeast or another system).

In some embodiments, the phytase may be a fungal phytase derived from Aspergillus niger, Aspergillus oryzae, Aspergillus fecuum, Aspergillus awamori, Aspergillus nidulans, Aspergillus fumigatus, Aspergillus terreus, Peniophora lycii, Cladosporium sp., Myceliophtora thermophila, Talaromyces thermophilus, Thermomyces lanuginosus, or Mucor pusillus. In other embodiments, the phytase may be a yeast phytase derived from Saccharomyces spp., such as Saccharomyces cerevisiae, Kluyveromyces spp., such as Kluyveromyces lactis, Arxula adeninivorans, Candida Krusei, Pichia anomala, or Schwanniomyces castillii. In still other embodiments, the phytase may be a bacterial phytase derived from Bacillus sp., such as Bacillus subtilis, Pseudomonas sp., such as Pseudomonas syringae, Escherichia coli, Selenomonas sp., Mitsuokella multiacidus, Citrobacter braaki, Obesumbacterium proteus, Klebsiella spp., or Shewanella oneidensis. In additional embodiments, the phytase may be a protozoan phytase derived from Paramecium tetraurelia. In further embodiments, the phytase may be a plant phytase derived from Avena sativa (oats), Hordeum vulgare (barley), Oryza sativa (rice), Secale cereale (rye), Sorghum bicolor, Triticum aestivum, Triticum durum, Triticum spelta (wheat species), Glycine max (soybean), Zea mays (corn), or Lilium spp. (lilies). In specific embodiments, the phytase may be of fungal, yeast, or bacterial origin.

In general, the amount of phytase in the composition may range from about 6 ppm to about 1000 ppm. In certain embodiments, the amount of phytase in the composition may be about 6, about 8, about 10, about 25, about 50, about 100, about 150, about 200, about 250, about 300, about 350, about 400, about 450, about 500, about 550, about 600, about 650, about 700, about 750, about 800, about 850, about 900, about 950, or about 10000 ppm. In specific embodiments, the amount of phytase in the composition may range from about 8 ppm to about 800 ppm.

Phytase activity is expressed in phytase units (FTU). One phytase unit (FTU) is defined as the amount of enzyme that liberates 1 micromole of inorganic phosphorus per minute from 0.0051 mol/l sodium phytate at 370 and pH 5.50 under the conditions of the test.

The amount of phytase administered in the composition can and will vary depending upon the type of animal and age of the animal. In an embodiment, the amount of phytase administered to the animal may be from about 100 to about 1500 FTU per kg of animal feed. In a further embodiment, the amount of phytase administered to the animal may be from about 200 to about 1200, about 300 to about 1000, about 400 to about 900 FTU per kg. In other embodiments, the amount of phytase administered to the animal may be at least about 100, about 150, about 200, about 250, about 300, about 350, about 400, about 450, about 500, about 550, about 600, about 650, about 700, about 750, about 800, about 850, about 900, about 950, about 1000 FTU per kg of animal feed or more. In an exemplary embodiment, the amount of phytase administered to the animal may be about 500 to 1000 FTU per kg of animal feed. In one preferred embodiment, the poultry diet further comprises one or more additional ingredients selected from the group consisting of a coccidiostat (Elanco, Coban 90), 500 FTU/kg of Phytase (AB Vista, Quantum Blue 5G), an NSPase (AB Vista, Econase XTP) and copper.

### NSPase

Non-starch polysaccharide (NSP) enzymes, also known as NSPases, are a group of enzymes that break down polysaccharides and help animals and humans digest and absorb nutrients. NSPases are commonly used in poultry feed to improve nutrient utilization by breaking down complex carbohydrates that are typically indigestible by the bird's own enzymes

In general, the amount of NSPase in the composition may range from about 6 ppm to about 1000 ppm. In certain embodiments, the amount of phytase in the composition may be about 6, about 8, about 10, about 25, about 50, about 100, about 150, about 200, about 250, about 300, about 350, about 400, about 450, about 500, about 550, about 600, about 650, about 700, about 750, about 800, about 850, about 900, about 950, or about 10000 ppm. In specific embodiments, the amount of NSPase in the composition may range from about 8 ppm to about 800 ppm.

NSPases, measured in EPU (Enzyme Activity Units), refer to non-starch polysaccharide degrading enzymes which are commonly used in animal feed to break down complex plant fibers like hemicellulose, primarily consisting of xylan and beta-glucan, improving nutrient digestibility by releasing trapped nutrients within the plant cell walls; the “E” in EPU stands for “enzyme” and signifies the activity level of the enzyme in breaking down these polysaccharides.

In an embodiment, the amount of NSPase in the composition may range from about 100 to about 3000 EPU per kg of feed. In a further embodiment, the amount of NSPase in the composition may be about 100 to about 1500 EPU per kg of animal feed. In a further embodiment, the amount of NSPase administered to the animal may be from about 200 to about 1200, about 300 to about 1000, about 400 to about 900 EPU per kg. In other embodiments, the amount of NSPase administered to the animal may be at least about 100, about 150, about 200, about 250, about 300, about 350, about 400, about 450, about 500, about 550, about 600, about 650, about 700, about 750, about 800, about 850, about 900, about 950, about 1000, about 1100, about 1200, about 1400 EPU per kg of animal feed or more. In an exemplary embodiment, the amount of NSPase administered to the animal may be about 1000 to 2000 EPU per kg of animal feed. In an exemplary embodiment, the amount of NSPase in the composition may range from about 1400 to about 1600 EPU per kg of composition.

### Copper

In some embodiments, the animal feeds and supplements comprising a low dose formulation of zinc and manganese from a covalently bonded source heretofore designated as a hydroxy chloride trace mineral source (HTM), also comprise copper.

In general, the amount of copper ion in the compositions may range from about 1 ppm to about 300 ppm. In some embodiments, the amount of copper ion in the composition may range from about 1 ppm to about 300 ppm. In a further embodiment, the amount of copper ion in the composition may be about 1, about 5, about 10, about 25, about 50, about 75, about 100, about 125, about 150, about 175, about 200, about 225, about 250, about 275, or about 300 ppm. In an exemplary embodiment, the amount of copper ion in the composition may range from about 10 to about 250 ppm. In a further embodiment, the amount of copper ion in the composition may range from about 100 to about 200 ppm. In one embodiment, the copper is provided as 125 ppm of copper in the form of tri basic copper chloride (Chemlock Nutrition, Nutrilock® 58% TBCC). (Aviagen, 2022).

In another embodiment, amino acid ratios are controlled and followed the genetic and USA industry standards.

In another embodiment, trace mineral premixes are included at 0.100% of the complete feed and mixed with a common basal in a standing Hobart mixer for 15 minutes for incorporation prior to large scale mixing and pelleting of each treatment.

The essentiality, absorption, bioavailability of trace minerals sourced from different forms discussed above (i.e., ITM, OTM and HTM) are woven into common poultry feed practices for the benefits that are well known to those versed in the art of poultry husbandry. Unfortunately, overfeeding of ionic trace minerals (ITM) trace minerals is routinely practiced in the poultry industry without gains in animal performance and this potentially contributes to trace mineral environmental concerns as the unused minerals excrete. Moreover, recommendations continue to push for increases in ITM sourced minerals according to allied company research and new genetic guidelines. (Aviagen, 2022) Our efforts focused on elements of the poultry industry feed methodology practicum. It was discovered that low dose zinc and manganese at a fixed copper and phytase levels generated statistically significant performance characteristics. Furthermore, it was discovered that supplementing lower levels of trace minerals sourced from HTM sources provided improved performance as measured by bird life performance, processing yields, tibia ash content and overall feed cost.

The current NRC recommended levels for copper, zinc and manganese are listed below in Table 1.

TABLE 3
Trace element feed recommendations

| Copper | | | Zinc | | | Manganese | | |
|---|---|---|---|---|---|---|---|---|
| (mg/kg feed/growth phase) | | | (mg/kg feed/growth phase) | | | (mg/kg feed/growth phase) | | |
| 16 | 16 | 16 | 120 | 120 | 120 | 120 | 120 | 120 |

The additive in the invention may assume any physical form that is appropriate to be mixed into poultry feed, such as powder, granules, bran, pellets, etc.

Another aspect of the invention refers to materials and methods that provide for animal or livestock feeds and supplements and particularly to animal feeds and supplements comprising a low dose formulation of zinc and manganese from a covalently bonded source heretofore designated as a hydroxy chloride trace mineral source (HTM), which leads to improved health in animals.

In some embodiments, the methods of providing animal feeds and supplements comprising a low dose formulation of zinc and manganese in poultry that is characterized by including the bird-feeding stage with feed that includes the additive described above, continuously or discontinuously, until the day of slaughter. In some embodiments, a feed comprising a low dose formulation of zinc and manganese is fed to poultry during the starter phase. In some embodiments, a feed comprising a low dose formulation of zinc and manganese is fed to poultry during the grower phase. In some embodiments, a feed comprising a low dose formulation of zinc and manganese is fed to poultry during both the starter and the grower phase. In some embodiments, a feed comprising a low dose formulation of zinc and manganese is fed to poultry daily for the first 5 days, 10 days, 15 days, 18 days, or 21 days, 28 days, 30 days, or 35 days after hatching. In some embodiments, a feed comprising a low dose formulation of zinc and manganese is fed to poultry for at least 50% of the days during the first 5 days, 10 days, 15 days, 18 days, or 21 days, 28 days, 30 days, or 35 days after hatching. In some embodiments, a feed comprising a low dose formulation of zinc and manganese is fed to poultry for at least 75% of the days during the first 5 days, 10 days, 15 days, 18 days, or 21 days, 28 days, 30 days, or 35 days after hatching. The day that poultry is slaughtered is typically between 30 and 60 days, particularly more than 40 days from the date chicks are hatched.

### Additional Components

In one embodiment, the premix or supplement may comprise a bioactive agent. Examples of suitable bioactive agents include vitamins, minerals, amino acids or amino acid analogs, antioxidants, organic acids, poly unsaturated fatty acids, essential oils, enzymes, prebiotics, probiotics, herbs, pigments, approved antibiotics, or combinations thereof.

In some embodiments, the bioactive agents may be one or more vitamins. Suitable vitamins include vitamin A, vitamin B1 (thiamine), vitamin B2 (riboflavin), vitamin B3 (niacin), vitamin B5 (pantothenic acid), vitamin B6 (pyridoxine), vitamin B7 (biotin), vitamin B9 (folic acid), vitamin B12, vitamin C, vitamin D, vitamin E, vitamin K, other B-complex vitamins (e.g., choline, carnitine, adenine), or combinations thereof. The form of the vitamin may include salts of the vitamin, derivatives of the vitamin, compounds having the same or similar activity of a vitamin, and metabolites of a vitamin.

In further embodiments, the bioactive agent may be one or more amino acids. Non-limiting suitable amino acids include standard amino acids (i.e., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine), non-standard amino acids (e.g., L-DOPA, GABA, 2-aminobutyric acid, and the like), amino acid analogs, or combinations thereof.

In alternate embodiments, the bioactive agent may be one or more antioxidants. Specific antioxidants that are useful as additional components in the additive to poultry feed, in accordance with the invention, are polyphenols, vitamins, minerals and their mixtures. Specific sources of polyphenols are grape seed extract, onion extract, rosemary extract and their mixtures. Specific vitamins that are useful for the invention are vitamins C and E and their mixtures. Specific minerals that are useful for the invention are selenium, zinc, manganese, copper and their mixtures.

In still other embodiments, the bioactive agent may be one or more organic acids. The organic acid may be a carboxylic acid or a substituted carboxylic acid. The carboxylic acid may be a mono-, di-, or tri-carboxylic acid. In general, the carboxylic acid may contain from about one to about twenty-two carbon atoms. Suitable organic acids, by way of non-limiting example, include acetic acid, adipic acid, butanoic acid, benzoic acid, cinnamaldehyde, citric acid, formic acid, fumaric acid, glutaric acid, glycolic acid, lactic acid, malic acid, mandelic acid, propionic acid, sorbic acid, succinic acid, tartaric acid, or combinations thereof. Salts of organic acids comprising carboxylic acids are also suitable for certain embodiments. Representative suitable salts include the ammonium, magnesium, calcium, lithium, sodium, potassium, selenium, iron, copper, and zinc salts of organic acids.

In yet other embodiments, the bioactive agent may be one or more poly unsaturated fatty acids. Suitable poly unsaturated fatty acids (PUFAs) include long chain fatty acids with at least 18 carbon atoms and at least two carbon-carbon double bonds, generally in the cis-configuration. In specific embodiments, the PUFA may be an omega fatty acid. The PUFA may be an omega-3 fatty acid in which the first double bond occurs in the third carbon-carbon bond from the methyl end of the carbon chain (i.e., opposite the carboxyl acid group). Suitable examples of omega-3 fatty acids include all-cis 7,10,13-hexadecatrienoic acid; all-cis-9,12,15-octadecatrienoic acid (alpha-linolenic acid, ALA); all-cis-6,9,12,15,-octadecatetraenoic acid (stearidonic acid); all-cis-8,11,14,17-eicosatetraenoic acid (eicosatetraenoic acid); all-cis-5,8,11,14,17-eicosapentaenoic acid (eicosapentaenoic acid, EPA); all-cis-7,10,13,16,19-docosapentaenoic acid (clupanodonic acid, DPA); all-cis-4,7,10,13,16,19-docosahexaenoic acid (docosahexaenoic acid, DHA); all-cis-4,7,10,13,16,19-docosahexaenoic acid; and all-cis-6,9,12,15,18,21-tetracosenoic acid (nisinic acid). In an alternative embodiment, the PUFA may be an omega-6 fatty acid in which the first double bond occurs in the sixth carbon-carbon bond from the methyl end of the carbon chain. Examples of omega-6 fatty acids include all-cis-9,12-octadecadienoic acid (linoleic acid); all-cis-6,9,12-octadecatrienoic acid (gamma-linolenic acid, GLA); all-cis-11,14-eicosadienoic acid (eicosadienoic acid); all-cis-8,11,14-eicosatrienoic acid (dihomo-gamma-linolenic acid, DGLA); all-cis-5,8,11,14-eicosatetraenoic acid (arachidonic acid, AA); all-cis-13,16-docosadienoic acid (docosadienoic acid); all-cis-7,10,13,16-docosatetraenoic acid (adrenic acid); and all-cis-4,7,10,13,16-docosapentaenoic acid (docosapentaenoic acid). In yet another alternative embodiment, the PUFA may be an omega-9 fatty acid in which the first double bond occurs in the ninth carbon-carbon bond from the methyl end of the carbon chain, or a conjugated fatty acid, in which at least one pair of double bonds are separated by only one single bond. Suitable examples of omega-9 fatty acids include cis-9-octadecenoic acid (oleic acid); cis-11-eicosenoic acid (eicosenoic acid); all-cis-5,8,11-eicosatrienoic acid (mead acid); cis-13-docosenoic acid (erucic acid), and cis-15-tetracosenoic acid (nervonic acid). Examples of conjugated fatty acids include 9Z,11 E-octadeca-9,11-dienoic acid (rumenic acid); 10E,12Z-octadeca-9,11-dienoic acid; 8E,10E,12Z-octadecatrienoic acid (α-calendic acid); 8E,10E,12E-octadecatrienoic acid (β-Calendic acid); 8E,10Z,12E-octadecatrienoic acid (jacaric acid); 9E,11E,13Z-octadeca-9,11,13-trienoic acid (α-eleostearic acid); 9E,11E,13E-octadeca-9,11,13-trienoic acid (0-eleostearic acid); 9Z,11Z,13E-octadeca-9,11,13-trienoic acid (catalpic acid), and 9E,11Z,13E-octadeca-9,11,13-trienoic acid (punicic acid).

In still other embodiments, the bioactive agents may be one or more probiotics or prebiotics. Probiotics and prebiotics include agents derived from yeast or bacteria that promote good digestive health. By way of non-limiting example, yeast-derived probiotics and prebiotics include yeast cell wall derived components such as β-glucans, arabinoxylan isomaltose, agarooligosaccharides, lactosucrose, cyclodextrins, lactose, fructooligosaccharides, laminariheptaose, lactulose, β-galactooligosaccharides, mannanoligosaccharides, raffinose, stachyose, oligofructose, glucosyl sucrose, sucrose thermal oligosaccharide, isomalturose, caramel, inulin, and xylooligosaccharides. In an exemplary embodiment, the yeast-derived agent may be β-glucans and/or mannanoligosaccharides. Sources for yeast cell wall derived components include Saccharomyces bisporus, Saccharomyces boulardii, Saccharomyces cerevisiae, Saccharomyces capsularis, Saccharomyces delbrueckii, Saccharomyces fermentati, Saccharomyces lugwigii, Saccharomyces microellipsoides, Saccharomyces pastorianus, Saccharomyces rosei, Candida albicans, Candida cloaceae, Candida tropicalis, Candida utilis, Geotrichum candidum, Hansenula americana, Hansenula anomala, Hansenula wingei, and Aspergillus oryzae. Probiotics and prebiotics may also include bacteria cell wall derived agents such as peptidoglycan and other components derived from gram-positive bacteria with a high content of peptidoglycan. Exemplary gram-positive bacteria include Lactobacillus acidophilus, Bifedobact thermophilum, Bifedobat longhum, Streptococcus faecium, Bacillus pumilus, Bacillus subtilis, Bacillus licheniformis, Lactobacillus acidophilus, Lactobacillus casei, Enterococcus faecium, Bifidobacterium bifidium, Propionibacterium acidipropionici, Propionibacteriium freudenreichii, and Bifidobacterium pseudolongum.

In alternate embodiments, the bioactive agent may be one or more enzymes or enzyme variants. Suitable non-limiting examples of enzymes include amylases, carbohydrases, cellulases, esterases, galactonases, galactosidases, glucanases, hemicellulases, hydrolases, lipases, oxidoreductases, pectinases, peptidases, phosphatases, phospholipases, phytases, proteases, transferases, xylanases, or combinations thereof.

In further embodiments, the bioactive agent may be one or more herbals. Suitable herbals and herbal derivatives, as used herein, refer to herbal extracts, and substances derived from plants and plant parts, such as leaves, flowers, and roots, without limitation. Non-limiting exemplary herbals and herbal derivatives include agrimony, alfalfa, aloe vera, amaranth, angelica, anise, barberry, basil, bayberry, bee pollen, birch, bistort, blackberry, black cohosh, black walnut, blessed thistle, blue cohosh, blue vervain, boneset, borage, buchu, buckthorn, bugleweed, burdock, capsicum, cayenne, caraway, cascara sagrada, catnip, celery, centaury, chamomile, chaparral, chickweed, chicory, chinchona, cloves, coltsfoot, comfrey, cornsilk, couch grass, cramp bark, culver's root, cyani, cornflower, damiana, dandelion, devils claw, dong quai, echinacea, elecampane, ephedra, eucalyptus, evening primrose, eyebright, false unicorn, fennel, fenugreek, figwort, flaxseed, garlic, gentian, ginger, ginseng, golden seal, gotu kola, gum weed, hawthorn, hops, horehound, horseradish, horsetail, hoshouwu, hydrangea, hyssop, iceland moss, irish moss, jojoba, juniper, kelp, lady's slipper, lemon grass, licorice, lobelia, mandrake, marigold, marjoram, marshmallow, mistletoe, mullein, mustard, myrrh, nettle, oatstraw, oregon grape, papaya, parsley, passion flower, peach, pennyroyal, peppermint, periwinkle, plantain, pleurisy root, pokeweed, prickly ash, psyllium, quassia, queen of the meadow, red clover, red raspberry, redmond clay, rhubarb, rose hips, rosemary, rue, safflower, saffron, sage, St. John's wort, sarsaparilla, sassafras, saw palmetto, scullcap, senega, senna, shepherd's purse, slippery elm, spearmint, spikenard, squawvine, stillingia, strawberry, taheebo, thyme, uva ursi, valerian, violet, watercress, white oak bark, white pine bark, wild cherry, wild lettuce, wild yam, willow, wintergreen, witch hazel, wood betony, wormwood, yarrow, yellow dock, yerba santa, yucca, or combinations thereof.

In yet other embodiments, the bioactive agent may be one or more antibiotics approved for use in livestock and poultry (i.e., antibiotics not considered critical or important for human health). Non-limiting examples of approved antibiotics include bacitracin, carbadox, ceftiofur, enrofloxacin, florfenicol, laidlomycin, linomycin, oxytetracycline, roxarsone, tilmicosin, tylosin, and virginiamycin.

### Animal Feed Premixes or Supplements

The compositions may be formulated into powders, pellets, liquids, crumbles, mash, etc.

An additional aspect of the present disclosure encompasses a feed premix or supplement, or an animal feed ration comprising the compositions defined above. The feed ration may be formulated to meet the nutritional requirements of a variety of animals.

Another aspect of the present disclosure comprises an animal feed premix or feed supplement comprising the compositions described herein. Typically, the premix will be added to various formulations of feed to formulate an animal feed ration. As will be appreciated by the skilled artisan, the particular premix or supplement can and will vary depending upon the feed ration and animal that the feed ration will be fed to. Accordingly, the premix or supplement may comprise a composition described herein.

A further aspect of the present disclosure encompasses an animal feed ration comprising the compositions described herein or as a premix or supplement as described above.

Feed ingredients that may be utilized in the present disclosure to satisfy an animal's maintenance energy requirements may include feed ingredients that are commonly provided to animals for consumption. Examples of such feed ingredients include grains, forage products, feed meals, feed concentrates, and the like.

Suitable grains include corn, corn gluten meal, soybeans, soybean meal, wheat, barley, oats, sorghum, rye, rice, and other grains, and grain meals.

Feed concentrates are feedstuffs that are high in energy and low in crude fiber. Concentrates also include a source of one or more ingredients that are used to enhance the nutritional adequacy of a feed supplement mix, such as vitamins and minerals.

The feed may be supplemented with a fat source. Non-limiting fats include plant oils, fish oils, animal fats, yellow grease, fish meal, oilseeds, distillers' grains, or combinations thereof. The fat source will generally comprise from about 1% to about 10% of the dry mass of the total feed ration, more preferably from about 2% to about 6%, and most preferably from about 3% to about 4%.

Feed rations of the present disclosure typically are formulated to meet the nutrient and energy demands of a particular animal. The nutrient and energy content of many common animal feed ingredients have been measured and are available to the public. The National Research Council has published books that contain tables of common ruminant feed ingredients and their respective measured nutrient and energy content. Additionally, estimates of nutrient and maintenance energy requirements are provided for growing and finishing cattle according to the weight of the cattle. National Academy of Sciences, Nutrient Requirements of Beef Cattle, Appendix Tables 1-19, 192-214, (National Academy Press, 2000); Nutrient Requirements of Dairy Cattle (2001), which are each incorporated herein by their entirety. This information can be utilized by one skilled in the art to estimate the nutritional and maintenance energy requirements of animal and determine the nutrient and energy content of animal feed ingredients.

### Carriers

The feed supplement of the present invention may be used in combination with other components or carriers.

Suitable carriers for feed enzymes include maltodextrin, limestone (calcium carbonate), cyclodextrin, wheat or a wheat component, sucrose, starch, anti-foam, Na₂SO₄, Talc, PVA and mixtures thereof. In addition there are a number of encapsulation techniques including those based on fat/wax coverage, adding plant gums etc.

Examples of other components include one or more of: thickeners, gelling agents, emulsifiers, binders, crystal modifiers, sweeteners (including artificial sweeteners), rheology modifiers, stabilizers, anti-oxidants, dyes, enzymes, carriers, vehicles, excipients, diluents, lubricating agents, flavoring agents, coloring matter, suspending agents, disintegrants, granulation binders etc. These other components may be natural. These other components may be prepared by use of chemical and/or enzymatic techniques.

As used herein the term “thickener or gelling agent” as used herein refers to a product that prevents separation by slowing or preventing the movement of particles, either droplets of immiscible liquids, air or insoluble solids.

The term “stabilizer” as used here is defined as an ingredient or combination of ingredients that keeps a product (e.g. a food product) from changing over time.

The term “emulsifier” as used herein refers to an ingredient (e.g. a food product ingredient) that prevents the separation of emulsions.

As used herein the term “binder” refers to an ingredient (e.g. a food ingredient) that binds the product together through a physical or chemical reaction.

The term “crystal modifier” as used herein refers to an ingredient (e.g. a food ingredient) that affects the crystallization of either fat or water.

“Carriers” or “vehicles” mean materials suitable for compound administration and include any such material known in the art such as, for example, any liquid, gel, solvent, liquid diluent, solubilizer, or the like, which is non-toxic and which does not interact with any components of the composition in a deleterious manner.

Examples of nutritionally acceptable carriers include, for example, grain, water, salt solutions, alcohol, silicone, waxes, petroleum jelly, vegetable oils, and the like.

Examples of excipients include one or more of: microcrystalline cellulose and other celluloses, lactose, sodium citrate, calcium carbonate, dibasic calcium phosphate, glycine, starch, milk sugar and high molecular weight polyethylene glycols.

Examples of disintegrants include one or more of: starch (preferably corn, potato or tapioca starch), sodium starch glycollate, croscarmellose sodium and certain complex silicates.

Examples of granulation binders include one or more of: polyvinylpyrrolidone, hydroxypropylmethylcellulose (HPMC), hydroxypropylcellulose (HPC), sucrose, maltose, gelatin and acacia.

Examples of lubricating agents include one or more of: magnesium stearate, stearic acid, glyceryl behenate and talc.

Examples of diluents include one or more of: water, ethanol, propylene glycol and glycerin, and combinations thereof.

The other components may be used simultaneously (e.g. when they are in admixture together or even when they are delivered by different routes) or sequentially (e.g. they may be delivered by different routes).

As used herein the term “component suitable for animal or human consumption” means a compound which is or can be added to the composition of the present invention as a supplement which may be of nutritional benefit, a fiber substitute or have a generally beneficial effect to the consumer.

By way of example, the components may be prebiotics such as alginate, xanthan, pectin, locust bean gum (LBG), inulin, guar gum, galacto-oligosaccharide (GOS), fructo-oligosaccharide (FOS), lactosucrose, soybean oligosaccharides, palatinose, isomalto-oligosaccharides, gluco-oligosaccharides and xylo-oligosaccharides.

### EXAMPLES

Example 1. In the current invention, sets of broilers were fed different levels of zinc (25, 50, or 75 ppm) and two levels of manganese (37.5, 75 ppm) sourced from HTMs. Each feed regiment with trace metal level are listed in Table 2 below. At the same time another set of broilers were fed 100 ppm sourced from ITMs, namely zinc sulfate and 120 ppm manganese also sourced from ITMs, namely manganese sulfate. Another set of broilers were fed 75 ppm zinc sourced from an alternative source of HTMs and 75 ppm manganese also sourced from an alternative source of HTMs. After 14, 28, 42 and 49 days of the feeding regiment described in tables 1-4, measurements for body weight gain, feed to gain and feed intake. After 49 days, cumulative mortality was also measured.

TABLE 4
Diet treatment levels of the trace mineral elements

| | | Birds per | Number | Birds per |
|---|---|---|---|---|
| Treatment | Description | replicate | of replicates | treatment |
| T1 | Industry ITM (Sulfates) | 23 | 12 | 276 |
| | (100 ppm Zn, 120 ppm Mn) | | | |
| T2 | Hydroxychloride Source A | 23 | 12 | 276 |
| | Control 75 ppm Zn, 75 ppm Mn | | | |
| T3 | Nutrilock ® 75 ppm Zn, 75 ppm | 23 | 12 | 276 |
| | Mn | | | |
| T4 | Nutrilock ® 75 ppm Zn, 37.5 ppm | 23 | 12 | 276 |
| | Mn | | | |
| T5 | Nutrilock ® 50 ppm Zn, 75 ppm | 23 | 12 | 276 |
| | Mn | | | |
| T6 | Nutrilock ® 50 ppm Zn, Mn 37.5 | 23 | 12 | 276 |
| | ppm | | | |
| T7 | Nutrilock ® 25 ppm Zn, 75 ppm | 23 | 12 | 276 |
| | Mn | | | |
| T8 | Nutrilock ® 25 ppm Zn, 37.5 ppm | 23 | 12 | 276 |
| | Mn | | | |

The diets and feeding phases are summarized in tables 3 and 4 below.

TABLE 5
Feed phase method parameters

| | Day Zero | Day 14 | Day 28 | Day 42 |
|---|---|---|---|---|
| Period | to Day 14 | to Day 28 | to Day 42 | to Day 49 |
| Quantity | Ad Libitum | Ad Libitum | Ad Libitum | Ad Libitum |
| Feed Type | Starter | Grower | Finisher | Withdrawal |
| Physical | Crumble* | Pellets** | Pellets** | Pellets** |
| Presentation | | | | |

*Mash diets from each phase were pelleted with a 170° F. conditioning for 30 seconds.
**Pellet diameter is 3/16 of an inch (4.76 mm) and between ¼ to ⅜ of an inch (6.36 to 9.53 mm)

TABLE 6
Feed phase method parameters

| | Zero to | 14 to | 28 Days | 42 Days |
|---|---|---|---|---|
| Parameters | 14 Days | 28 Days | to 42 Days | to 49 Days |
| Digestible | 1.18% | 1.04% | 0.94% | 0.90% |
| Lysine | | | | |
| Apparent | | | | |
| Metabolizable | | | | |
| Energy | 1370 | 1400 | 1420 | 1430 |
| (corrected for | | | | |
| Nitrogen | | | | |
| retention) | | | | |
| Total Calcium | 0.92% | 0.84% | 0.76% | 0.70% |
| Available | 0.47% | 0.43% | 0.39% | 0.36% |
| Phosphorous | | | | |

Body weight, body weight gain, feed to gain, and feed intake in broilers fed different trace mineral premixes from 0 to 14 days of age is shown in Table 5. It was discovered that the low dose methodology generated live body weight, body weight gain, feed to gain and feed intake data that was significantly improved to the ITM sulfate control and statistically equivalent to the HTM A control supplementation levels.

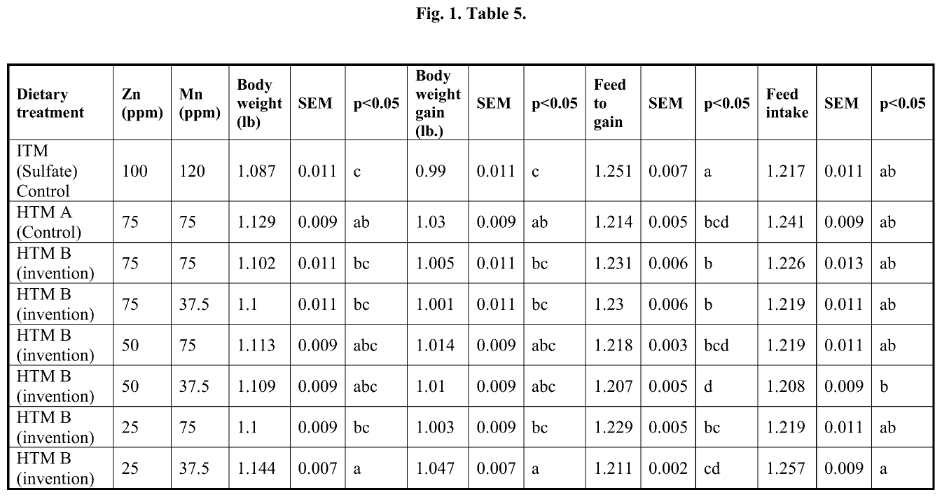

Table 5 is Shown in FIG. 1: Zero to 14 day growth stage data

FIG. 1 is Table 7 showing Zero to 14 day growth stage data: (The values are means±SEM, n=12 replicate pens with 23 birds per pen for each dietary treatment. Values within a section with different superscripts for a given parameter differ, (P<0.05).

Body weight, body weight gain, feed to gain, and feed intake in broilers fed different trace mineral premixes from 0 to 28 days of age is shown in Table 6. It was discovered that the low dose methodology generated live body weight and body weight gain data that was significantly improved to the ITM sulfate control and statistically equivalent to the HTM A control supplementation levels.

TABLE 8
Body weight, body weight gain, and feed efficiency of broilers fed different trace mineral premixes from 0-28 days
of age (¹The values are means ± SEM, n = 12 replicate pens with 23 birds per pen for each dietary treatment.

| | | | | | | Body | | | | | | |
|---|---|---|---|---|---|---|---|---|---|---|---|---|
| | | | Body | | | weight | | | | | | |
| Dietary | Zn | Mn | weight | | | gain | | | Feed to | | Feed | |
| treatment | (ppm) | (ppm) | (lb) | SEM | p < 0.05 | (lb.) | SEM | p < 0.05 | gain | SEM | intake | SEM |
| ITM | 100 | 120 | 4.118 | 0.037 | b | 4.019 | 0.037 | b | 1.405 | 0.006 | 5.518 | 0.053 |
| (Sulfate) | | | | | | | | | | | | |
| Control | | | | | | | | | | | | |
| HTM A | 75 | 75 | 4.204 | 0.024 | ab | 4.105 | 0.024 | ab | 1.395 | 0.003 | 5.611 | 0.033 |
| (Control) | | | | | | | | | | | | |
| HTM B | 75 | 75 | 4.151 | 0.04 | ab | 4.054 | 0.04 | ab | 1.407 | 0.004 | 5.626 | 0.055 |
| (invention) | | | | | | | | | | | | |
| HTM B | 75 | 37.5 | 4.193 | 0.04 | ab | 4.094 | 0.04 | ab | 1.407 | 0.005 | 5.666 | 0.035 |
| (invention) | | | | | | | | | | | | |
| HTM B | 50 | 75 | 4.167 | 0.029 | ab | 4.07 | 0.029 | ab | 1.402 | 0.005 | 5.573 | 0.06 |
| (invention) | | | | | | | | | | | | |
| HTM B | 50 | 37.5 | 4.149 | 0.037 | ab | 4.05 | 0.037 | ab | 1.391 | 0.004 | 5.6 | 0.042 |
| (invention) | | | | | | | | | | | | |
| HTM B | 25 | 75 | 4.142 | 0.026 | ab | 4.045 | 0.026 | ab | 1.392 | 0.008 | 5.54 | 0.053 |
| (invention) | | | | | | | | | | | | |
| HTM B | 25 | 37.5 | 4.27 | 0.044 | a | 4.171 | 0.044 | a | 1.396 | 0.004 | 5.69 | 0.046 |
| (invention) | | | | | | | | | | | | |

a-b Values within a section with different superscripts for a given parameter differ, (P < 0.05).

Body weight, body weight gain, feed to gain, and feed intake in broilers fed different trace mineral premixes from 0 to 42 days of age is shown in Table 7. It was discovered that the low dose methodology generated live body weight and body weight gain data that was equivalent to the ITM sulfate control and the HTM A control supplementation levels.

TABLE 9
Body weight, body weight gain, and feed efficiency of broilers fed different
trace mineral premixes from 0-42 days of age (Values are means ± SEM,
n = 12 replicate pens with 23 birds per pen for each dietary treatment).

| | | | | | Body | | | | | |
|---|---|---|---|---|---|---|---|---|---|---|
| | | | Body | | weight | | | | | |
| Dietary | Zn | Mn | weight | | gain | | Feed to | | Feed | |
| treatment | (ppm | (ppm | (lb) | SEM | (lb) | SEM | gain | SEM | intake | SEM |
| ITM | 100 | 120 | 7.802 | 0.086 | 7.705 | 0.086 | 1.572 | 0.007 | 11.612 | 0.139 |
| (Sulfate) | | | | | | | | | | |
| Control | | | | | | | | | | |
| HTM A | 75 | 75 | 7.91 | 0.086 | 7.813 | 0.086 | 1.571 | 0.006 | 11.861 | 0.117 |
| (Control) | | | | | | | | | | |
| HTM B | 75 | 75 | 7.932 | 0.075 | 7.833 | 0.075 | 1.573 | 0.008 | 11.925 | 0.192 |
| (invention) | | | | | | | | | | |
| HTM B | 75 | 37.5 | 8.036 | 0.106 | 7.939 | 0.106 | 1.574 | 0.008 | 12 | 0.101 |
| (invention) | | | | | | | | | | |
| HTM B | 50 | 75 | 7.963 | 0.079 | 7.866 | 0.079 | 1.573 | 0.005 | 11.678 | 0.13 |
| (invention) | | | | | | | | | | |
| HTM B | 50 | 37.5 | 7.796 | 0.134 | 7.699 | 0.134 | 1.576 | 0.009 | 11.729 | 0.159 |
| (invention) | | | | | | | | | | |
| HTM B | 25 | 75 | 7.769 | 0.115 | 7.672 | 0.115 | 1.569 | 0.009 | 11.572 | 0.152 |
| (invention) | | | | | | | | | | |
| HTM B | 25 | 37.5 | 7.813 | 0.066 | 7.714 | 0.066 | 1.574 | 0.008 | 11.715 | 0.126 |
| (invention) | | | | | | | | | | |

Body weight, body weight gain, feed to gain, and feed intake in broilers fed different trace mineral premixes from 0 to 49 days of age is shown in Table 8. It was discovered that the low dose methodology generated live body weight and body weight gain data that was equivalent to the ITM sulfate control and the HTM A control supplementation levels.

TABLE 10
Body weight, body weight gain, and feed efficiency of broilers fed different trace mineral premixes from 0-49
days of age. Values are means ± SEM, n = 12 replicate pens with 23 birds per pen for each dietary treatment.

| | | | | | Body | | | | | | | |
|---|---|---|---|---|---|---|---|---|---|---|---|---|
| | | | Body | | weight | | Feed | | | | | |
| Dietary | Zn | Mn | weight | | gain | | to | | Feed | | | Mortality |
| treatment | (ppm) | (ppm) | (lb.) | SEM | (lb.) | SEM | gain | SEM | intake | SEM | Mortality % | No# |
| ITM | 100 | 120 | 9.149 | 0.112 | 9.052 | 0.11 | 1.695 | 0.01 | 14.82 | 0.17 | 4.71 | 13 |
| (Sulfate) | | | | | | | | | | | | |
| Control | | | | | | | | | | | | |
| HTM A | 75 | 75 | 9.356 | 0.128 | 9.257 | 0.13 | 1.679 | 0.01 | 15.06 | 0.17 | 4.348 | 12 |
| (Control) | | | | | | | | | | | | |
| HTM B | 75 | 75 | 9.493 | 0.128 | 9.396 | 0.13 | 1.672 | 0.01 | 15.08 | 0.22 | 1.812 | 5 |
| (invention) | | | | | | | | | | | | |
| HTM B | 75 | 37.5 | 9.57 | 0.126 | 9.471 | 0.13 | 1.673 | 0.01 | 15.21 | 0.16 | 3.623 | 10 |
| (invention) | | | | | | | | | | | | |
| HTM B | 50 | 75 | 9.398 | 0.143 | 9.299 | 0.14 | 1.675 | 0.01 | 14.87 | 0.17 | 4.71 | 13 |
| (invention) | | | | | | | | | | | | |
| HTM B | 50 | 37.5 | 9.116 | 0.196 | 9.017 | 0.2 | 1.697 | 0.01 | 14.84 | 0.22 | 3.261 | 9 |
| (invention) | | | | | | | | | | | | |
| HTM B | 25 | 75 | 9.158 | 0.15 | 9.059 | 0.15 | 1.689 | 0.01 | 14.61 | 0.2 | 4.71 | 13 |
| (invention) | | | | | | | | | | | | |
| HTM B | 25 | 37.5 | 9.213 | 0.11 | 9.114 | 0.11 | 1.684 | 0.01 | 14.79 | 0.16 | 3.623 | 10 |
| (invention) | | | | | | | | | | | | |

Coefficient of variation in live body weight in broilers fed different trace mineral premixes from 0 to 49 days of age is shown in Table 9. It was discovered that the low dose methodology generated live body weight data that was equivalent to the ITM sulfate control, HTM A control and the HTM B supplementation levels.

TABLE 11
Coefficient of variation in live body weight in broilers fed
different trace mineral premixes from 0 to 49 days of age.

| | | | Coefficient of |
|---|---|---|---|
| | Zn | Mn | Variation at |
| Dietary treatment | (ppm) | (ppm) | Day 49 |
| ITM (Sulfate) | 100 | 120 | 8.73 ± 0.48 |
| Control | | | |
| HTM A (Control) | 75 | 75 | 7.87 ± 0.66 |
| HTM B (invention) | 75 | 75 | 7.54 ± 0.42 |
| HTM B (invention) | 75 | 37.5 | 7.29 ± 0.62 |
| HTM B (invention) | 50 | 75 | 7.90 ± 0.48 |
| HTM B (invention) | 50 | 37.5 | 7.78 ± 0.46 |
| HTM B (invention) | 25 | 75 | 8.09 ± 0.67 |
| HTM B (invention) | 25 | 37.5 | 8.51 ± 0.40 |

The tibia ash percent in broilers fed different trace mineral premixes from 0 to 49 days of age is shown in Table 10. It was discovered that the low dose feed methodology generated equivalent or better tibia weight and tibia ash.

TABLE 12
Tibia ash percent in broilers fed different trace
mineral premixes from 0 to 49 days of age.

| | Zn | Mn | | | |
|---|---|---|---|---|---|
| Dietary treatment | (ppm) | (ppm) | % Tibia weight | % Tibia ash | p < 0.05 |
| ITM (Sulfate) | 100 | 120 | 0.331 ± 0.015 | 35.804 ± 0.288 | b |
| Control | | | | | |
| HTM A (Control) | 75 | 75 | 0.332 ± 0.011 | 36.597 ± 0.277 | ab |
| HTM B (invention) | 75 | 75 | 0.336 ± 0.023 | 36.838 ± 0.396 | ab |
| HTM B (invention) | 75 | 37.5 | 0.322 ± 0.017 | 37.224 ± 0.520 | ab |
| HTM B (invention) | 50 | 75 | 0.331 ± 0.017 | 36.862 ± 0.444 | ab |
| HTM B (invention) | 50 | 37.5 | 0.335 ± 0.013 | 36.622 ± 0.363 | ab |
| HTM B (invention) | 25 | 75 | 0.323 ± 0.015 | 37.500 ± 0.439 | a |
| HTM B (invention) | 25 | 37.5 | 0.325 ± 0.011 | 36.944 ± 0.219 | ab |

Processing yields from 50-day old broilers fed different trace mineral premixes from 0 to 49 days of age is shown below in table 11. It was discovered that no significant differences occurred in processing weight between the treatments and the low dose feeding methodology was equivalent to the current industry regimens (ITM control, HTM A control).

TABLE 13
Processing yields from 50-day old broilers fed different trace mineral premixes.

| | | | Live | | Fasted | | | |
|---|---|---|---|---|---|---|---|---|
| | | | weight | | weight | | | |
| Dietary | Zn | Mn | (day 49 | | (day 50 | SE | Loss of | SE |
| treatment | ppm | ppm | of age) | SEM | of age) | M | weight, %2 | M |
| ITM (Sulfate) | 100 | 120 | 9.18 | 0.1146 | 8.7325 | 0.11 | 4.97 | 0.37 |
| Control | | | | | | | | |
| HTM A (Control) | 75 | 75 | 9.385 | 0.1344 | 8.8868 | 0.12 | 5.12 | 0.44 |
| HTM B | 75 | 75 | 9.513 | 0.1168 | 8.9573 | 0.11 | 5.6 | 0.42 |
| (invention) | | | | | | | | |
| HTM B | 75 | 37.5 | 9.564 | 0.1344 | 9.0058 | 0.11 | 6.01 | 0.47 |
| (invention) | | | | | | | | |
| HTM B | 50 | 75 | 9.414 | 0.1455 | 8.8758 | 0.1 | 5.6 | 0.44 |
| (invention) | | | | | | | | |
| HTM B | 50 | 37.5 | 9.081 | 0.2116 | 8.6134 | 0.19 | 5.01 | 0.4 |
| (invention) | | | | | | | | |
| HTM B | 25 | 75 | 9.187 | 0.1499 | 8.7082 | 0.12 | 5.23 | 0.45 |
| (invention) | | | | | | | | |
| HTM B | 25 | 37.5 | 9.202 | 0.1146 | 8.7016 | 0.1 | 5.28 | 0.22 |
| (invention) | | | | | | | | |

TABLE 14
Processing yields from 50-day old broilers fed different
trace mineral premixes from 0 to 49 days of age.

| | | | Hot | | Chilled | | | |
|---|---|---|---|---|---|---|---|---|
| Dietary | Zn | Mn | carcass | | carcass | | Frame | |
| treatment | (ppm) | (ppm) | lb. | % | lb. | % | lb. | % |
| ITM | 100 | 120 | 6.737 ± | 77.2± | 6.717± | 76.9± | 1.419± | 16.3 ± |
| (Sulfate) | | | 0.084 | 0.15 | 0.088 | 0.21 | 0.017 | 0.11 |
| Control | | | | | | | | |
| HTM A | 75 | 75 | 6.860 ± | 77.2± | 6.832± | 76.9± | 1.470 ± | 16.6± |
| (Control) | | | 0.094 | 0.17 | 0.092 | 0.13 | 0.019 | 0.16 |
| HTM B | 75 | 75 | 6.924 ± | 77.3 ± | 6.893 ± | 77.0± | 1.461 ± | 16.3 |
| (invention) | | | 0.083 | 0.13 | 0.083 | 0.13 | 0.015 | 0.10 |
| HTM B | 75 | 37.5 | 6.942 ± | 77.1± | 6.915 ± | 76.8± | 1.483 ± | 16.5 ± |
| (invention) | | | 0.079 | 0.19 | 0.077 | 0.25 | 0.022 | 0.12 |
| HTM B | 50 | 75 | 6.851 ± | 77.2 | 6.818 ± | 76.8± | 1.450 | 16.3 ± |
| (invention) | | | 0.083 | 0.18 | 0.077 | 0.23 | 0.019 | 0.12 |
| HTM B | 50 | 37.5 | 6.618 ± | 76.9± | 6.591 ± | 76.6± | 1.419 ± | 16.5 ± |
| (invention) | | | 0.138 | 0.13 | 0.138 | 0.22 | 0.028 | 0.13 |
| HTM B | 25 | 75 | 6.728 ± | 77.3 ± | 6.715 ± | 77.1± | 1.446 ± | 16.6± |
| (invention) | | | 0.094 | 0.19 | 0.083 | 0.30 | 0.017 | 0.14 |
| HTM B | 25 | 37.5 | 6.746 ± | 77.5 ± | 6.697 ± | 77.0 | 1.410± | 16.2± |
| (invention) | | | 0.081 | 0.13 | 0.077 | 0.13 | 0.022 | 0.22 |

The data from processing yields from 50 day broilers fed different trace mineral premixes from 0-49 days of age. It was discovered that no significant differences occurred in total carcass yield or further processing yield (Pectoralis Major, Minor, Total White Meat, and Leg Quarters, Frame) between the treatments and the low dose feeding methodology was equivalent to the current industry regimens (ITM control, HTM A control).

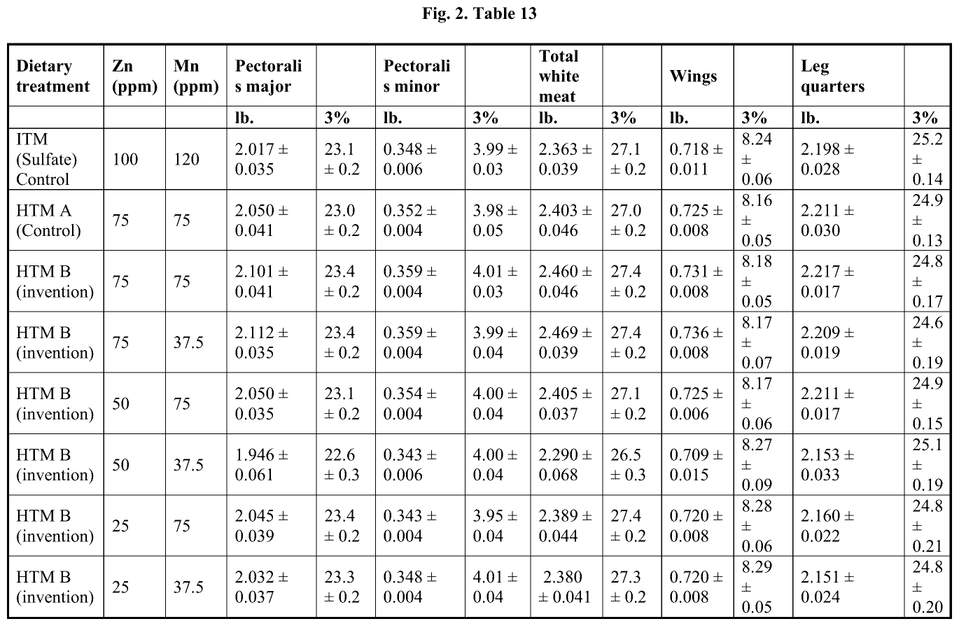

Table 13 is Shown in FIG. 2.

FIG. 2 shows Table 15: Processing yields from 50 day broilers fed different trace mineral premixes from 0-49 days of age.

### REFERENCES

Aviagen. (2022).
Beattie, J. H., & Kwun, I. S. (2004). Is zinc deficiency a risk factor in atherosclerosis?. British Journal of Nutrition, 91, 177-181.
Berg, J. M. (1990). Zinc fingers and other metal-binding domains: elements for interactions between molecules. Journal of Biological Chemistry, 265, 6513-6516.
Brugger, D., & Windisch, W. (2015). Environmental responsibilities of livestock feeding using trace mineral supplements. Anim Nutr., 1:113-118.
Cohen, J. (2014). Trace Minerals—Exploring the New Developments in swine and ruminants. Journal of Animal Sci.; 92, 2, 414-415.
Cousins, R. J., Liuzzi, J. P., & Lichten, L. A. (2006). Mammalian zinc transport, trafficking and signals. Journal of Biological Chemistry, 281, 24085-24089.
Dibner, J. J., & Richards, J. D. (2005). Mineral metabolism and chelated minerals for hatchlings. In G. P. J., Recent Advances in Animal Nutrition (p. 390). Nottingham, U.K.: Nottingham University Press.
Dibner, J. J., Richards, J. D., Kitchell, M. L., & Quiroz, M. A. (2007). Metabolic challenges and early bone development. The Journal ofApplied Poultry Research, 16, 126-137.
dos Santos, T. S., Augusto, K. V., Han, Y., Sartori, M. M., Batistioli, J. S., Neto, A. C., . . . Sartori, J. R. (2023). Effects of dietary copper and zinc hydroxychloride supplementation on bone development, skin quality and hematological parameters of broilers chickens. J Anim Physiol Anim Nutr., 107:1241-1250.
Gregory, E. M., & Fridovich, I. (1974). Superoxide dismutases: properties, distribution, and functions. In W. S. Hoekstra, Trace Element Metabolism in Animals—2 (pp. 486-488.). Baltimore, Maryland: University Park Press.
Holubiev, M., Holubieva, T., & Sychov, M. (2020). Effect of supplemental Mn, Zn, Fe and Cu and their interactions on the. Ukrainian Journal of Veterinary and Agricultural Sciences, Vol. 3, N 3.
Inventors [symbol] (2021). CN113598281A, Production method of coated slow-release microelement premix for poultry.
Inventors: [symbol] , A. π. (2020). WO2020159398A1: Complete integrated microelement feed additive based on organic compounds of iron, manganese, zinc, copper, cobalt, iodine, and selenium, and method for application of same.
Leeson, S. (2009). Copper metabolism and dietary needs. World's Poultry Science Journal, 65, 353-366.
López-Alonso, M. (2012). Trace minerals and livestock: not too much not too little. ISRN Vet Sci., 1-18.
Lu, W. B., Kuang, Y. G., Ma, Z. X., & Liu, Y. G. (2020). The Effect of Feeding Broiler with Inorganic, Organic and Coated Trace Minerals on Performance, Economics and Retetention of Copper and Zinc. Journal of Appl. Poultry. Res., 1084-1090.
Olukosi, O. A., van Kuijk, S., & Han, Y. (2018). Copper and zinc sources and levels of zinc inclusion influence growth performance, tissue trace mineral content, and carcass yield of broiler chickens. Poultry Science, 97:3891-3898.
Olukosi, O. A., van Kuijk, S., & Han, Y. (2018). Copper and zinc sources and levels of zinc inclusion influence growth performance, tissue trace mineral content, and carcass yield of broiler chickens. Poultry Science, 97:3891-3898.
Pang, Y., & Applegate, T. J. (2007). Effects of dietary copper supplementation and copper source on digesta pH, calcium, zinc, and copper complex size in the gastrointestinal tract of the broiler chicken. Poultry Science, 86, 531-537.
Prasad, A. S. (1967). The Importance of Zinc in Human Nutrition. The American Journal of Clinical Nutrition, 20, 6, 648-652.
Scrutton, M. C., Utter, M. F., & Mildvan, A. S. (1966). Pyruvate carboxylase: The presence of tightly boundmanganese. Journal of Biological Chemistry, 241, 3480-3487.
St Clair, D. (2004). Manganese superoxide dismutase: genetic variation and regulation. Journal of Nutrition, 134, 3190S-3191S.
Suttle, N. (2010). Mineral Nutrition in Livestock, 4th Ed. (ISBN 978-1-84593-472-9). Oxfordshire, UK: CABI International.
Underwood, E. (1977). Trace Elements in Human and Animal Nutrition, 4th Ed. London: Academic Press.
Weller, M., Overton, T., Rourke, J., & Armstrong, F. (2014). Inorganic chemistry, vol. 6. Oxford: Oxford University Press.
Yenice, E., Mizrak2, C., Gülteki, M., & Tunca, M. (2015). Effects of Organic and Inorganic Forms of Manganese, Zinc, Copper, and Chromium on Bioavailability of These Minerals and Calcium in Late-Phase Laying Hens. Biol Trace Elem Res (2015), 167:300-307.
Zelenka, J. (2013). Allometric growth of copper, zinc, manganese and iron in slow- and fast-growing young chickens. Acta Universitatis Agriculturae et Silviculturae Mendelianae Brunensis, LXI, No. 1, pp. 237-241.

While the disclosure has been described with reference to exemplary embodiments, it will be understood by those skilled in the art that various changes may be made, and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure is not limited to the particular embodiments disclosed for carrying out this disclosure, but that the disclosure will include all embodiments falling within the scope of the appended claims. Moreover, the use of the terms first, second, etc. do not denote any order or importance, but rather the terms first, second, etc. are used to distinguish one element from another.

The description of the present disclosure has been presented for purposes of illustration and description but is not intended to be exhaustive or limited to the disclosure in the form disclosed. Many modifications and variations will be apparent to those of ordinary skill in the art without departing from the scope of the disclosure. The described embodiments were chosen and described in order to best explain the principles of the disclosure and the practical application, and to enable others of ordinary skill in the art to understand the disclosure for various embodiments with various modifications as are suited to the particular use contemplated.

What is claimed is:

1. A method for feeding poultry comprised of a low dose supplementation of a common basal with major bulk ingredients of corn and soybean meal with a 3% fixed inclusion in all phases of meat and bone meal feed using trace metals from hydroxy metal chloride mineral sources over the full course of a normal growth to harvest period such that at least one key industry process indicator is substantially improved.

2. A method for feeding poultry of claim 1, wherein the key industry process indicator is selected from the group consisting of body weight, feed to gain, leg quality, harvest (yield) quantities and cost.

3. A method for feeding poultry of claim 1, wherein the poultry is selected from the group consisting of broiler chickens, laying chickens and turkeys.

4. A method of feeding poultry of claim 1 wherein the growth stage is from zero to 49 days for chickens and zero to 20 weeks for turkeys.

5. A method for feeding poultry of claim 1, wherein the trace metals from hydroxy metal chloride sources are copper (Cu), zinc (Zn) and manganese (Mn).

6. A method for feeding poultry of claim 1 wherein the level of Zn is maintained at a level of 1-30 ppm on total weight of feed.

7. A method for feeding poultry of claim 1, wherein the level of Zn is maintained at a level of 25 ppm on total weight of feed.

8. The method for feeding poultry of claim 1, wherein the level of Mn is maintained at a level of 1-40 ppm on total weight of feed.

9. A method for feeding poultry of claim 1, wherein the level of Mn is maintained at a level of 37.5 ppm on total weight of feed.

10. A method for feeding poultry of claim 1, wherein the composition further comprises at least one feed or food ingredient selected from the group consisting of proteins, carbohydrates, fats, probiotics, probiotics, enzymes, vitamins, immune modulators, milk replacers, minerals, amino acids, coccidiostats, acid-based products, and medicines.

11. A method for feeding poultry of claim 1, wherein the level of copper is maintained at a level of from about 100 to about 200 ppm based on total weight of feed.

12. A method for feeding poultry of claim 1, wherein the level of phytase is maintained at a level of from about 500 to about 1000 FTU/kg.

13. A method for feeding poultry of claim 1 wherein, amino acid ratios followed the genetic and USA industry standards.

14. A method of feeding poultry of claim 1, wherein feed to gain was improved 3.3% at 14 days of age.

15. A method of feeding poultry of claim 1, wherein body weight gain was improved 5.7% at 14 days of age.

16. A method of feeding poultry of claim 1, wherein tibia ash was improved 3.18% at 49 days of age.

17. A method of feeding poultry of claim 1, wherein average feed cost was reduced 0.1%.
